(19) United States
(12) Patent Application Publication
(10) Pub. No.: US 2023/0381222 A1
(43) Pub. Date: Nov. 30, 2023

(54) LYOPHILIZED PHARMACEUTICAL COMPOSITIONS OF COPPER HISTIDINATE

(71) Applicant: ZYDUS LIFESCIENCES LIMITED, Ahmedabad (IN)

(72) Inventors: Kannan Essakimuthu Muthaiyyan, Ahmedabad (IN); Debjani Manoj Singh, Ahmedabad (IN); Tushar Surajmal Nahata, Ahmedabad (IN); Pradeep Jawarchand Chouhan, Ahmedabad (IN)

(21) Appl. No.: 18/325,249

(22) Filed: May 30, 2023

(30) Foreign Application Priority Data
May 31, 2022 (IN) 202221031124

(51) Int. Cl. A61K 33/34; A61K 9/19; A61K 31/4172

(52) U.S. Cl. CPC A61K 33/34; A61K 9/19; A61K 31/4172

(57) ABSTRACT

The present invention relates to lyophilized pharmaceutical compositions of copper histidinate and one or more pharmaceutically acceptable excipients. It also relates to processes for preparing such compositions.

### FIELD OF THE INVENTION

The present invention relates to lyophilized pharmaceutical compositions of copper histidinate. The invention also relates to processes for preparing such compositions.

### BACKGROUND OF THE INVENTION

Copper is an essential nutrient for the body. Together with iron, it enables the body to form red blood cells. It helps maintain healthy bones, blood vessels, nerves, and immune function, and it contributes to iron absorption. Copper deficiency is rare and causes both hematological and neurological diseases. Copper deficiency can also result from a rare genetic disorder called Menkes disease. Researchers have tried to develop various formulations containing copper to treat copper deficiency.

Sherwood et al., Journal of Inherited Metabolic Disease, 12 Suppl., 2, 393-396 (1989), discloses liquid formulation of copper histidinate.

A book “Small molecule therapy for genetic disease” by Cambridge University press (ISBN 978-0-521-51781-2) in Chapter No. 14 named, “Small copper complexes for treatment of acquired and inherited copper deficiency syndromes”, by Stephen G. Kaler, pages 202-212, discloses a freeze-dried product of copper histidine.

Basak et al. (Journal of Inorganic Biochemistry, 51 (1-2), page no. 415 (1993)), discloses that, in order to circumvent the stability problems associated in solution, copper-histidine was formulated in freeze-dried form. It also discloses that the presence of sodium chloride causes aggregations during lyophilization resulting in a turbid solution when reconstituted with sterile water. It also discloses that, the freeze-dried copper-histidine with no additives is by far the best as reflected by the stability studies at different storage conditions. Sarkar et al., The Journal of Pediatrics, 123 (5), 828-30 (1993), cites B as ak et al., and reiterates the same.

Kaler et al., Biochemical and Molecular Medicine, 57, 37-46 (1996), discloses copper histidine prepared as a freeze-dried product for Menkes disease. It also discloses that sterile water was used to dissolve copper histidine prior to freeze-drying, and 0.9% normal saline was used to resuspend the freeze-dried product.

International (PCT) Publication No. WO/2010/042102 discloses preparation and administration of copper histidine. It also discloses a process for dissolving copper chloride dihydrate, L-histidine, and sodium hydroxide in water to prepare a solution and freeze drying the solution to obtain freeze-dried product. It also discloses reconstitution of freeze-dried product with 0.9% sodium chloride injection.

There is still a need for alternate lyophilized pharmaceutical compositions of copper histidinate having improved stability and meeting other requirements as set forth in the description below.

### SUMMARY OF THE INVENTION

In one general aspect, the present invention provides a lyophilized pharmaceutical composition comprising copper histidinate, wherein the lyophilized pharmaceutical composition, for example, a lyophilized powder or cake, has improved parameters, such as good aesthetic appearance.

Embodiments of the lyophilized pharmaceutical composition may include one or more of the following features. The composition may further comprise one or more pharmaceutically acceptable excipients, for example, one or more lyoprotectants, one or more pH adjusting agents, optionally one or more antioxidants, optionally one or more buffering agents, and optionally one or more preservatives.

The lyoprotectant may be mixed with copper histidinate solution to provide a pre-lyophilization solution and then the solution may be lyophilized to provide a lyophilized composition comprising copper histidinate and a lyoprotectant. Copper histidinate present in the pre-lyophilization solution may be formed in-situ upon mixing of copper(II) chloride dihydrate solution and histidine solution.

In another general aspect, the present invention provides a process for preparing a lyophilized pharmaceutical composition of copper histidinate, wherein the process comprises the steps of:
(a) preparing copper(II) chloride dihydrate solution,
(b) preparing histidine solution,
(c) mixing the solutions obtained at steps (a) and (b),
(d) adding one or more lyoprotectants to the solution obtained at step (c), (e) adding one or more pH adjusting agents to have a pH of solution to about 7.0-7.5, and
(f) performing lyophilization (freeze drying) of the solution obtained at step (e).

In another general aspect, the present invention provides a glass vial/SiO₂ vial/quartz vial, a glass ampoule, a glass bottle, a plastic bottle, a plastic bag, a glass/plastic pre-filled syringe, or a glass/plastic single/dual chamber cartridge containing a lyophilized pharmaceutical composition comprising copper histidinate and one or more lyoprotectants.

In another general aspect, the present invention provides a method of treating Menkes disease in a patient in need thereof, comprising administering to the patient a solution comprising copper histidinate, one or more pharmaceutically acceptable excipients, and a vehicle, wherein the administration is through a subcutaneous route.

The details of one or more embodiments of the invention are set forth in the description below. Other features, objects and advantages of the invention will be apparent from the description.

### BRIEF DESCRIPTION OF THE FIGURES

FIG. 1 depicts composition of the invention

FIG. 2 depicts composition of the comparative example

### DETAILED DESCRIPTION OF THE INVENTION

The inventors of the present invention have found that when a lyoprotectant, for example, sodium chloride, is used for preparing a lyophilized pharmaceutical composition of copper histidinate, the composition having improved parameters can be obtained. The lyophilized pharmaceutical composition, for example, a lyophilized powder or cake, having improved parameters, such as good aesthetic appearance (lyophilized powder or cake essentially free from any aggregates, lumps, and/or lyophilization defects, viz., melt back, collapsed cake, shrinkage of cake, slanted cake, etc.) can be obtained.

The inventors have also found that when the lyophilized pharmaceutical composition is mixed with sterile water for injection, a clear solution (reconstituted) having improved physical parameters can be obtained. This also allows usage of sterile water for injection as a reconstitution vehicle and avoids dependency on saline solution as a reconstitution vehicle. The reconstituted solution having improved physical parameters, such as solution being clear (not hazy or turbid/without any visible particles/free of any particles) can be obtained. Such a clear solution being essentially free from any particulate matter is suitable for administration through a parenteral route to a human being in need thereof, for example, a patient in need thereof.

In one embodiment, the present invention provides a lyophilized pharmaceutical composition comprising copper histidinate and one or more pharmaceutically acceptable excipients (additives). The lyophilized pharmaceutical composition of the invention is a lyophilized powder or cake for reconstitution which is essentially free from any lyophilization defects, for example, melt back, collapsed cake, shrinkage of cake, slanted cake. The lyophilized powder or cake is essentially free from any aggregates, lumps, and/or lyophilization defects.

Examples of pharmaceutically acceptable excipients may include, but not limited to, lyoprotectants, pH adjusting agents, antioxidants, buffering agents, and preservatives.

Examples of suitable lyoprotectants may include, but are not limited to, sodium chloride, mannitol, sucrose, dextrose, lactose, and cyclodextrin derivatives (hydroxypropyl-beta-cyclodextrin, sulphobutylether-beta-cyclodextrin, etc.), or any combination thereof. For example, sodium chloride is a suitable lyoprotectant. The lyophilized pharmaceutical composition of the invention, upon mixing with sterile water for injection, provides a clear solution, wherein the sodium chloride is in a concentration of between about 1 mg/mL and about 18 mg/mL. The lyoprotectant may be mixed with copper histidinate solution to provide a pre-lyophilization solution and then the solution is lyophilized to provide a lyophilized composition comprising copper histidinate and a lyoprotectant. Copper histidinate present in the pre-lyophilization solution may be formed in-situ upon mixing of copper(II) chloride dihydrate solution and L-histidine solution. The lyophilized powder or cake of the present invention comprising lyoprotectants have good aesthetic appearance, for example, essentially free from any aggregates, lumps, and/or lyophilization defects, viz., melt back, collapsed cake, shrinkage of cake, slanted cake, etc. The presence of lyoprotectant decreases the collapse temperature, for example, presence of sodium chloride decreases the collapse temperature from about −70° C. to about −27° C. and allows efficient lyophilization. Additionally, the lyophilized powder or cake of the invention comprising sodium chloride upon reconstitution with suitable reconstitution vehicle, for example, sterile water for injection, provides a clear solution (not hazy or turbid/without any visible particles/free of any particles) which remains clear after storage for 24 hours at 100° C. The lyophilized powder or cake of the invention comprising pharmaceutically acceptable excipient/lyoprotectant/sodium chloride may have higher surface area compared to that of the lyophilized powder or cake without such excipient/lyoprotectant/sodium chloride.

The term “about” as used herein, refers to encompass +/−20%, 15%, 10%, 5%, 2%, 1%, or 0.25% of the numerical value of the number with which it is being used.

Examples of suitable pH adjusting agents may include, but are not limited to, sodium hydroxide, potassium hydroxide, hydrochloric acid, and phosphoric acid, or any combination thereof. Sodium hydroxide present in a lyophilized pharmaceutical composition is in a sufficient amount to produce such a clear solution upon mixing with sterile water for injection which has a pH value of between about 6.0 and about 8.0, for example, between about 7.0 and about 7.5.

Examples of suitable antioxidants may include, but are not limited to, monothioglycerol, ascorbic acid, 1-cysteine, sodium sulfite, sodium bisulfite, disodium edetate, butylated hydroxytoluene (BHT), butylated hydroxyanisole (BHA), propyl gallate, sodium ascorbate, erythorbic acid, potassium metabisulfite, propionic acid, sodium formaldehyde sulphoxylate, reduced glutathione, thiourea, n-acetylcysteine, methionine, alkyl gallate, including propyl gallate, vitamin E, or other tocopherol analogs, including tocopherol acetate or TPGS, or any combination thereof.

Examples of suitable buffering agents may include, but are not limited to, acetate buffer (e.g. sodium acetate and acetic acid etc.), phosphate buffer (e.g. monobasic sodium phosphate, dibasic sodium phosphate etc.), citrate buffer (e.g. anhydrous citric acid and trisodium citrate dihydrate etc.), carbonate buffer, lactate buffer, glycine buffer, borate buffer (boric acid/potassium chloride), tris buffer, tromethamine buffer, or any combination thereof.

Examples of suitable preservatives may include, but are not limited to, chlorobutanol, benzalkonium chloride, methyl paraben, propyl paraben, benzoic acid, sodium benzoate, sorbic acid, benzethonium chloride, cetyl pyridinium chloride, benzyl bromide, phenylmercury nitrate, phenylmercury acetate, thiomersal, merthiolate, chlorhexidine, phenylethyl alcohol, quaternary ammonium chloride, sodium benzoate, sodium propionate, or any combination thereof.

In one embodiment, the present invention provides a lyophilized pharmaceutical composition comprising copper histidinate, one or more lyoprotectants, and one or more pH adjusting agents. The lyophilized pharmaceutical composition may comprise copper histidinate, sodium chloride, and sodium hydroxide. The lyophilized pharmaceutical composition in each vial may comprise copper histidinate in an amount of between about 0.500 mg and about 10.000 mg, for example, between about 0.725 mg and about 6.100 mg, sodium chloride in an amount of between about 1 mg and about 18 mg, and sodium hydroxide in a sufficient amount to adjust the pH between about 7.0 and about 7.5.

In another embodiment, the lyophilized pharmaceutical composition comprising copper histidinate, sodium chloride, and sodium hydroxide, wherein the composition contains sodium chloride in an amount of between about 1 mg and about 18 mg, for example, 2 mg, 3 mg, 4 mg, 5 mg, 6 mg, 7 mg, 8 mg, 9 mg, 10 mg, 11, mg, 12 mg, 13, mg, 14 mg, mg, 16 mg, or 17 mg.

In another embodiment, the lyophilized pharmaceutical composition comprising copper histidinate, sodium chloride, and sodium hydroxide provides a weight ratio of copper histidinate to sodium chloride of between about 1:0.1 and about 1:7.0, for example, between about 1:0.3 and about 1:6.3. The lyophilized pharmaceutical composition retains at least about 90% of the potency of copper histidinate (% assay) in the pharmaceutical composition, as determined by Ultraviolet—visible spectroscopy, after storage for 1 month or more, for example, 6 months, 12 months, 18 months, 24 months, or 36 months at 50° C. and 80% RH, at 40° C. and 75% RH, at controlled room temperature (between about ° C. and about 25° C.) and 60% RH, at 2° C. to 8° C., or at −15° C. to −20° C. The lyophilized pharmaceutical composition can be supplied at storage condition either at 2° C. to 8° C. or at controlled room temperature.

In another embodiment, the lyophilized pharmaceutical composition comprising copper histidinate, sodium chloride, and sodium hydroxide has % water content (% by weight of the composition) not more than 4% immediately after its manufacturing (initial) as well as during its storage at various conditions. For example, the % water content (% by weight of the composition) is not more than about 4%, for example, not more than 3%, after storage of the lyophilized pharmaceutical composition for 1 month or more, for example, 6 months, 12 months, 18 months, 24 months, or 36 months at 40° C. and 75% RH, at 25° C. and 60% RH, or at 2° C. to 8° C. The % water content (% by weight of the composition) of the lyophilized pharmaceutical composition after storage for 6 months at 25° C. and 60% RH do not change/increase more than 5.0%, for example, 4.0%, 3.0%, or 2.0%, compared to that of the composition at an initial level. The % water content (% by weight of the composition) of the lyophilized pharmaceutical composition after storage for 6 months at 2° C. to 8° C. does not change/increase more than 5.0%, for example, 4.0%, 3.0%, or 2.0%, compared to that of the composition at an initial level. The determination of % water content can be performed by Karl Fischer Coulometer.

In one embodiment, the present invention provides a clear solution (reconstituted) upon mixing of the lyophilized pharmaceutical composition comprising copper histidinate, sodium chloride, and sodium hydroxide with sterile water for injection. The clear solution is provided in less than 60 seconds, for example, 45 seconds, 30 seconds, 15 seconds, or seconds, after the mixing. The clear solution comprises copper histidinate in a concentration of about 2.9 mg/mL, sodium chloride in a concentration of about 9 mg/mL, and sodium hydroxide in a sufficient amount to provide pH of between about 7.0 and about 7.5, which can be administered to a human being in need thereof without any further dilution. The administration can be through a subcutaneous injection route with a dose volume of about 0.5 mL of the clear solution (2.9 mg/mL of copper histidinate).

As used herein, the term “clear solution” means a solution which is essentially free from any visible particles/particulate matter that can be observed on visual inspection and shall comply with the following limit for sub-visible particulate matters: NMT 6000 particles/container of NMT 25 μm size and NMT 600 particles/container of NMT 10 μm size, as determined by laser light scattering microscopy. The clear solution may provide % transmittance, when measured at 650 nm, not less than 95%, for example, not less than 96%, not less than 97%, not less than 98%, not less than 99%, not less than 99.5%, not less than 99.6%, not less than 99.7% or not less than 99.8%. The clear solution may provide the value of absorbance not more than 1 AU, for example, not more then 0.75, 0.4, 0.3, 0.2, 0.1 or 0.05.

The clear solution of the invention may provide osmolality value of between about 300 mOsmol/kg and about 380 mOsmol/kg.

The clear solution (reconstituted solution) of the invention is suitable for administration through various parenteral routes, for example, intramuscular route (intradeltoid or intragluteal), subcutaneous route, or intravenous route.

In one embodiment, the lyophilized pharmaceutical composition comprising copper histidinate, sodium chloride, and sodium hydroxide may retain at least 90% of the copper histidinate (% assay), as determined by Ultraviolet—visible spectroscopy, after storage for more than 1 month, for example, 3 months, 6 months, 12 months, 18 months, 24 months, months, or 36 months, at 40° C. and 75% RH, controlled room temperature and 60% RH, 2° C. to 8° C., or at −15° C. to −20° C.

The lyophilized pharmaceutical composition does not contain total impurities more than 2.0%, for example, 1.5%, 1.0%, or 0.5%, by weight of copper histidinate, as determined by HPLC, after storage for 6 months, 12 months, 18 months, 24 months, 30 months, or 36 months at 40° C. and 75% RH, controlled room temperature and 60% RH, 2° C. to 8° C., or at −15° C. to −20° C. The lyophilized pharmaceutical composition does not contain 2-Oxo-L-Histidine more than 0.5%, for example, 0.2%, or 0.1%, by weight of copper histidinate, as determined by HPLC, after storage for 6 months, 12 months, 18 months, 24 months, 30 months, or 36 months at 40° C. and 75% RH, controlled room temperature and 60% RH, 2° C. to 8° C., or at −15° C. to −20° C.

In one embodiment, the reconstituted solution of the present invention remains clear immediately after preparing it as well as after its storage for 1 month at 25° C. and 60% RH and/or after its storage for 2 months at 2° C.-8° C.

In one embodiment, the present invention provides a process for preparing a lyophilized pharmaceutical composition comprising copper histidinate, sodium chloride, and sodium hydroxide, the process comprising the steps of:
(a) dissolving copper(II) chloride dihydrate in water, for example, sterile water for injection, to obtain copper(II) chloride dihydrate solution,
(b) dissolving L-histidine in sterile water for injection to obtain a histidine solution,
(c) mixing the solutions obtained at steps (a) and (b),
(d) adding a lyoprotectant to the solution obtained at step (c),
(e) adding a pH adjusting agent to have a solution of pH 7.0-7.5,
(f) filtering the solution obtained at step (e) using aseptic filtration,
(g) filling the solution obtained at step (f) in vials, and
(h) performing lyophilization of the vials obtained at step (g).

In another embodiment, the present invention provides a process for preparing a lyophilized pharmaceutical composition comprising copper histidinate, sodium chloride, and sodium hydroxide, the process comprising the steps of:
(a) dissolving lyophilized copper histidinate in sterile water for injection,
(b) adding a lyoprotectant to the solution obtained at step (a),
(c) adding a pH adjusting agent to have a solution of pH 7.0-7.5,
(d) filtering the solution obtained at step (c) using aseptic filtration,
(e) filling the solution obtained at step (d) in vials, and
(f) performing lyophilization of the vials obtained at step (e).

The lyophilized copper histidinate may be obtained by a process comprising the steps of:
(a) dissolving copper(II) chloride dihydrate in water, for example, sterile water for injection, to obtain copper(II) chloride dihydrate solution,
(b) dissolving L-histidine in water, for example, sterile water for injection, to obtain a histidine solution,
(c) mixing the solutions obtained at steps (a) and (b), and
(d) performing lyophilization of the solution obtained at step (c).

The lyophilized pharmaceutical composition may be suitable to undergo a sterilization process to provide a sterile composition. The examples of suitable sterilization process may include, but are not limited to, aseptic membrane filtration sterilization.

In one embodiment, the lyophilized pharmaceutical composition of the present invention is supplied/provided in a suitable container (primary packaging material), for example, in a glass vial/SiO₂ vial/quartz vial, a glass ampoule, a glass bottle, a plastic bottle, a glass/plastic pre-filled syringe, or a glass/plastic single/dual chamber cartridge.

In another embodiment, the lyophilized pharmaceutical composition of the present invention is supplied/provided, in a dual chamber injector device wherein the first chamber contains copper histidinate lyophilized powder and the second chamber contains sterile water for injection. At the time of drug administration to a human being in need thereof, for example, to a patient in need thereof, mixing of the content of both the chambers provide a clear solution. The dual chamber injector device may contain various constituent parts, for example, a glass or plastic barrel, a plunger, one or more rubber stoppers, a needle, and a cap.

In one embodiment, the present invention provides a method of treating Menkes disease comprising administering to a patient in need thereof a solution comprising copper histidinate, one or more pharmaceutically acceptable excipients, and a vehicle, wherein the administration is through a subcutaneous route. The subcutaneous administration provides 0.5 mL dose of 2.9 mg/mL of copper histidinate solution (1.45 mg copper histidinate). The solution is a reconstituted solution provided by reconstitution of the lyophilized pharmaceutical composition comprising copper histidinate and one or more pharmaceutically acceptable excipients (for example, lyoprotectants) in the vehicle (for example, sterile water for injection). The administration is through a parenteral route, for example, subcutaneous route, intravenous route, or intramuscular route.

In one embodiment, the lyophilized pharmaceutical composition of the present invention upon mixing with water, for example, sterile water for injection, provides a clear solution for administration through parenteral route, wherein upon administration to a human being in need thereof, for example, a patient in need thereof, the administration may provide a very mild pain intensity. The visual analog scale score (VAS score) is a tool to determine intensity of perceived pain. A human being/patient after administration, for example, after 1 minute of administration, may indicate his/her perceived pain intensity along a 100 mm horizontal line, where 0 means “no pain” and 100 means “unbearable pain”. The administration according to the present invention may provide a VAS score value below 80, for example, below 50, below 30, below 16, below 10, or 0.

In another embodiment, the present invention provides methods of treating low copper levels in blood plasma (copper deficiency) of a human being, for example a patient, comprising administering to a human being in need thereof, for example, a patient in need thereof, a solution comprising copper histidinate, one or more pharmaceutically acceptable excipients, and a vehicle, wherein the administration is through parenteral route. The solution is a reconstituted solution provided by reconstitution of the lyophilized pharmaceutical composition comprising copper histidinate and one or more pharmaceutically acceptable excipients (for example, lyoprotectants) in the vehicle (for example, sterile water for injection).

### Abbreviations

μm: Micrometer
mg: Milligram
mL Millilitre
RH: Relative Humidity
° C.: Degree Centigrade/Celsius
NMT: Not More Than
q.s.: Quantity Sufficient
AU: Absorbance Units
mOsm/kg: Milliosmoles Per Kilogram
HPLC: High-performance liquid chromatography
1M: 1 month
2M: 2 months

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Example 1

TABLE 1

| Sr. | | |
|---|---|---|
| no. | Ingredients | Quantity |
| 1 | Copper(II) chloride dihydrate | 1.345 mg/mL |
| 2 | L-Histidine | 2.450 mg/mL |
| 3 | Sodium chloride | 9.000 mg/mL |
| 4 | Sodium hydroxide | q.s. to pH |
| 5 | Sterile Water for Injection | q.s. to 1 mL |

### Process:

Solution of Copper(II) chloride dihydrate:
1. Copper(II) chloride dihydrate was added in water for injection.

### Solution of Histidine:

2. L-Histidine was added in water for injection.

Lyophilized Powder/Cake of Copper Histidinate:
3. Solution of copper(II) chloride dihydrate and solution of histidine were mixed.
4. Sodium chloride was added to the above solution obtained at step 3.
5. pH was adjusted to 7.0-7.5 using sodium hydroxide.
6. The solution obtained at step 5 was filtered using aseptic membrane filtration.
7. The solution obtained at step 6 was filled in glass vials.
8. The vials obtained at step 7 were lyophilized to obtain lyophilized powder/cake.

### Comparative Example

TABLE 2

| Sr. | | |
|---|---|---|
| no. | Ingredients | Quantity |
| 1 | Copper(II) chloride dihydrate | 1.345 mg/mL |
| 2 | L-Histidine | 2.450 mg/mL |
| 3 | Sodium hydroxide | q.s. to pH |
| 4 | Sterile Water for Injection | q.s. to 1 mL |

### Process:

Solution of Copper(II) chloride dihydrate:
1. Copper(II) chloride dihydrate was added in water for injection.

### Solution of Histidine:

2. L-Histidine was added in water for injection.

### Lyophilization:

3. Solution of copper(II) chloride dihydrate and solution of histidine were mixed.
4. pH was adjusted to 7.0-7.5 using sodium hydroxide.
5. The solution obtained at step 4 was filtered using aseptic membrane filtration.
6. The solution obtained at step 5 was filled in glass vials.
7. The vials obtained at step 6 were lyophilized.

The lyophilized compositions of Example 1 (step 8) and comparative example (step 7), were tested for their physical stability, and the results are reported in Table 3A below.

TABLE 3A

| | With sodium chloride | | | | | Without sodium chloride | | | |
|---|---|---|---|---|---|---|---|---|---|
| | | 25° C./ | 2-8° | 40° C./ | 50° C./ | | 25° C./ | | |
| | | 60% RH | C. | 75% RH | 80% RH | | 60% RH | 2-8° C. | |
| Test | T₀ | 6 M | 12 M | 6 M | 1 M | T₀ | 1 M | 1 M | 2 M |
| Description | * | * | * | * | * | ** | ** | ** | ** |

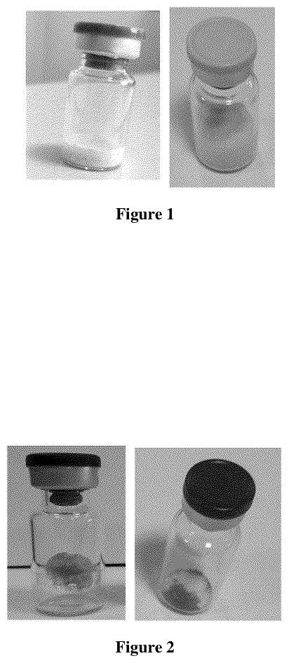

*Blue colored solid/cake/powder without any lyophilization defect (melt back, collapsed cake, shrinkage of cake, slanted cake) as shown in FIG. 1.
**Blue colored solid/cake/powder with one or more of the lyophilization defects (melt back, collapsed cake, shrinkage of cake, slanted cake) as shown in FIG. 2.

It is evident from a comparison of the physical stability data of lyophilized compositions of Example 1 (step 8) and comparative example (step 7), provided in the above Table 3A, that sodium chloride provides a better physical stability to the lyophilized composition, which is free of any lyophilization defects.

The lyophilized composition of Example 1 (step 8) was tested for its chemical stability, and the results are reported in Table 3B below.

TABLE 3B

| | | 25° C./ | 40º C./ |
|---|---|---|---|
| Test at 6M stability | 2-8° C. | 60% RH | 75% RH |
| % Assay (By Ultraviolet-visible | 113.90 | 112.00 | 113.00 |
| spectroscopy) | | | |
| 2-Oxo-L-Histidine | BQL | BQL | BQL |
| Total impurities | 0.0 | 0.0 | 0.0 |

BQL = Below Quantification Limit
2-Oxo-L-Histidine: 2-amino-3-(2-oxo-2,3-dihydro-1H-imidazol-4-yl) propanoic acid

The lyophilized compositions of Example 1 (step 8) and comparative example (step 7), were tested for % water content (% by weight of the composition) and the results are reported in Table 3C below, as determined by Karl Fischer Coulometer (Instrument: Metrohm 831 KF coulometer).

TABLE 3C

| | Storage/time | | Water | Water content (%) change |
|---|---|---|---|---|
| Composition | point | | content (%) | compared to initial |
| With sodium | T₀ | — | 0.89 | 0 |
| chloride | 25° C./ | 6M | 2.78 | 1.89 |
| | 60% RH | | | |
| | 2-8° C. | 6M | 2.65 | 1.76 |
| | 40° C./ | 6M | 2.84 | 1.95 |
| | 75% RH | | | |
| Without sodium | T₀ | — | 1.89 | 0 |
| chloride | 25° C./ | 6M | 9.01 | 7.12 |
| | 60% RH | | | |
| | 2-8° C. | 6M | 7.86 | 5.97 |
| | 40° C./ | 6M | 9.29 | 7.40 |
| | 75% RH | | | |

It is evident from a comparison of the % water content data for lyophilized compositions of Example 1 (step 8) and comparative example (step 7), provided in the above Table 3C, that (i) % water content (% by weight of the composition) of the lyophilized composition containing sodium chloride is not more than 4% immediately after its manufacturing (initial) as well as during its storage under various temperature/humidity conditions, and (ii) % water content (% by weight of the composition) of the lyophilized composition containing sodium chloride, after storage for 6 months at various conditions do not change/increase more than 5.0% compared to that of the composition at an initial level.

The lyophilized compositions of Example 1 (step 8) and comparative example (step 7), were mixed separately with a sufficient quantity of sterile water for injection to prepare two separate reconstituted solutions having a concentration of copper histidinate at about 2.9 mg/mL, and such reconstituted solutions were tested for physical and chemical parameters, and the results are reported in Table 3D below.

TABLE 3D
Reconstituted solution results (Reconstitution vehicle: Sterile Water for
injection)

| | With sodium | |
|---|---|---|
| Test | chloride | Without sodium chloride |
| Storage condition | 25° C./60% RH | 25° C./60% RH |
| | 1M | 1M |
| Description | Clear blue solution | Blue color solution with brown |
| | with no visible | tint, slightly hazy solution/with |
| | particle | particles |
| pH | 7.3 | 7.5 |
| Osmolality | 326 | 38 |
| % Assay (By | 96.8 | 91.9 |
| Ultraviolet- | | |
| visible spectroscopy) | | |
| Storage condition | 100° C. | 100° C. |
| | 24 hours | 24 hours |
| Description | Clear blue solution | Blue color solution with brown |
| | with no visible | tint, slightly hazy solution/with |
| | particle | particles |

It is evident from a comparison of the description for reconstituted solutions as reported in Table 3D above, that sodium chloride provides stability to the reconstituted solution.

We claim:

1. A lyophilized pharmaceutical composition comprising copper histidinate and one or more pharmaceutically acceptable excipients.

2. The lyophilized pharmaceutical composition according to claim 1, wherein the excipient is a lyoprotectant.

3. The lyophilized pharmaceutical composition according to claim 2, wherein the lyoprotectant is sodium chloride.

4. The lyophilized pharmaceutical composition according to claim 1, wherein the excipient is a pH adjusting agent.

5. The lyophilized pharmaceutical composition according to claim 4, wherein the pH adjusting agent is sodium hydroxide.

6. The lyophilized pharmaceutical composition according to claim 1, wherein the composition comprises about 0.500 mg to about 10.000 mg of copper histidinate.

7. The lyophilized pharmaceutical composition according to claim 3, wherein the sodium chloride is present in an amount of from about 1 mg to about 18 mg.

8. The lyophilized pharmaceutical composition according to claim 1, wherein the composition is a lyophilized powder or cake.

9. The lyophilized pharmaceutical composition according to claim 8, wherein the lyophilized powder or cake is essentially free from any aggregates, lumps, and/or lyophilization defects.

10. The lyophilized pharmaceutical composition according to claim 8, wherein the lyophilized powder or cake retains at least about 90% of the potency of copper histidinate (% assay) in the pharmaceutical composition, as determined by Ultraviolet—visible spectroscopy, after storage for 6 months at 2° C. to 8° C.

11. The lyophilized pharmaceutical composition according to claim 8, wherein the lyophilized powder or cake does not contain total impurities more than 2.0%, by weight of copper histidinate, as determined by HPLC.

12. A lyophilized pharmaceutical composition comprising copper histidinate and one or more pharmaceutically acceptable excipients, wherein the lyophilized pharmaceutical composition upon mixing with sterile water provides a clear solution.

13. The lyophilized pharmaceutical composition according to claim 12, wherein the solution remains clear after storage for 24 hours at 100° C.

14. The lyophilized pharmaceutical composition according to claim 12, wherein the solution remains clear after storage for 1 month at 25° C. and 60% RH.

15. The lyophilized pharmaceutical composition according to claim 12, wherein the solution has a pH of between about 7.0 and about 7.5.

16. The lyophilized pharmaceutical composition according to claim 12, wherein the solution contains about 2.9 mg/mL of copper histidinate.

17. The lyophilized pharmaceutical composition according to claim 16, wherein the solution is for administration through a subcutaneous route with a dose volume of about 0.5 mL of the solution.

18. A lyophilized pharmaceutical composition comprising copper histidinate and one or more pharmaceutically acceptable excipients, wherein the lyophilized pharmaceutical composition upon mixing with water provides a clear solution, and wherein the solution has osmolality value of from about 300 mOsm/kg to about 380 mOsm/kg.

19. A process for preparing a lyophilized pharmaceutical composition comprising copper histidinate, sodium chloride, and one or more pH adjusting agents, the process comprising the steps of:
(a) dissolving copper(II) chloride dihydrate in water to obtain copper(II) chloride dihydrate solution,
(b) dissolving L-histidine in water to obtain histidine solution,
(c) mixing the solutions obtained at steps (a) and (b),
(d) adding sodium chloride to the solution obtained at step (c),
(e) adding a pH adjusting agent to achieve pH of about 7.0-7.5,
(f) filling the solution obtained at step (e) in a container, and
(g) performing lyophilization of the container obtained at step (f).

20. A method of treating Menkes disease in a patient in need thereof, comprising administering to the patient the clear solution according to claim 12.
